(19) United States
(12) Patent Application Publication
(10) Pub. No.: US 2008/0064951 A1
(43) Pub. Date: Mar. 13, 2008

(54) Magnetic resonance imaging system

(75) Inventors: Shinichi Kitane, Nasushiobara-shi (JP); Yuichi Yamashita, Matsudo-shi (JP)

Correspondence Address: NIXON & VANDERHYE, PC, 901 NORTH GLEBE ROAD, 11TH FLOOR, ARLINGTON, VA, 22203, US

(73) Assignees: KABUSHIKI KAISHA TOSHIBA, Tokyo (JP); Toshiba Medical Systems Corporation, Tochigi (JP)

(21) Appl. No.: 11/898,255

(22) Filed: Sep. 11, 2007

(30) Foreign Application Priority Data
Sep. 13, 2006 (JP) 2006-248423
Aug. 10, 2007 (JP) 2007-209520

(51) Int. Cl. A61B 5/055

(52) U.S. Cl. 600/413

(57) ABSTRACT

A magnetic resonance imaging apparatus includes a collecting unit which collects magnetic resonance data regarding a subject by a predetermined pulse sequence, and a control unit which controls the collecting unit so that it collects the magnetic resonance data for at least a slice within an inhaling or an exhaling period of the subject.

### CROSS-REFERENCE TO RELATED APPLICATIONS

This application is based upon and claims the benefit of priority from prior Japanese Patent Applications No. 2006-248423, filed Sep. 13, 2006; and No. 2007-209520, filed Aug. 10, 2007, the entire contents of both of which are incorporated herein by reference.

### BACKGROUND OF THE INVENTION

1. Field of the Invention

The present invention is related to a magnetic resonance imaging apparatus suitable for imaging the abdomen of a subject.

2. Description of the Related Art

Previously, breath-holding imaging has been used when obtaining an image of the abdomen by the magnetic resonance imaging apparatus. However, the breath-holding imaging had major constraints in, such as, imaging conditions since the image can only be obtained within a time the subject is capable of holding his/her breath.

First and second conventional arts which are shown in, for example, FIGS. 7 and 8 are known as techniques to overcome these problems.

The first conventional art uses a spin echo (SE) method, which rearranges the encode data sequence in accordance with a breathing cycle to make artifact caused by movement less noticeable (refer to Japanese Patent Application KOKAI No. 9-182728).

The second conventional art carries out respiration synchronization. However, it does not rearrange data sequence as in the first conventional art and initiates imaging by trigger timing. After obtaining 1 phase encode (PE) data (real scan), to maintain a steady state, a sequence is run under the same imaging condition without collecting data (dummy scan). This way, the artifact caused by movement can be reduced while T1W contrast is maintained in a sequence of SE system.

In the first conventional art, as imaging time grew longer, there was a problem that a stable image could not be obtained due to difficulties of carrying out appropriate arrangement of the encode data consistently.

In the second conventional art, by using the SE system the dummy scan became unusable. Further, repeating time (TR) could not be varied. Therefore, even when it is time for trigger timing, proper collection can only be initiated at a timing where a term of 1TR is further terminated. This makes imaging difficult to be carried out at portions where movement is stable. Thus, there has been a problem that the movement artifact could not be suppressed sufficiently.

### BRIEF SUMMARY OF THE INVENTION

Due to the above circumstances, it has been desired to carry out imaging in high quality, which is unaffected by movement.

According to a first aspect of the present invention, there is provided a magnetic resonance imaging apparatus comprising: a collecting unit which collects magnetic resonance data regarding a subject by a predetermined pulse sequence; and a control unit which controls the collecting unit so that it collects the magnetic resonance data for at least a slice within an inhaling or an exhaling period of the subject.

According to a second aspect of the present invention, there is provided a magnetic resonance imaging apparatus comprising: a collecting unit which collects magnetic resonance data regarding a subject by a predetermined pulse sequence; and a control unit which controls the collecting unit so that it collects the magnetic resonance data for at least a slice within a period in which a heart of the subject has less variation in movement.

According to a third aspect of the present invention, there is provided a magnetic resonance imaging apparatus comprising: an obtaining unit which obtains a respiration signal which indicates respiration of a subject; a scan unit which carries out a three dimensional scan of the subject in synchronization with the obtained respiration signal; and a control unit which controls the scan unit so that it collects the magnetic resonance data for a slice encode of the three dimensional scan within an inhalation or an exhalation period based on the respiration signal.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a view for showing a configuration of a magnetic resonance imaging apparatus related to a first embodiment of the present invention.

FIG. 2 is a view for showing timing to implement data collecting operation and a pulse sequence for collecting data.

FIG. 3 is a view for showing an arrangement of obtained data in k-space.

FIG. 4 is a view for showing an example of a pulse sequence of an FFE method used with a water excitation method.

FIG. 5A is a view for showing an example of an image obtained by employing the sequence shown in FIG. 4.

FIG. 5B is a view for showing an example of an image obtained by breath-holding imaging in a pulse sequence according to an field echo (FE) method.

FIG. 6 is a view for showing an example of a sequence for applying an IR pulse prior to collecting data.

FIG. 7 is a view for showing characteristics of the first conventional art.

FIG. 8 is a view for showing characteristics of the second conventional art.

### DETAILED DESCRIPTION OF THE INVENTION

An embodiment of the present invention will be explained as follows in reference to the drawings.

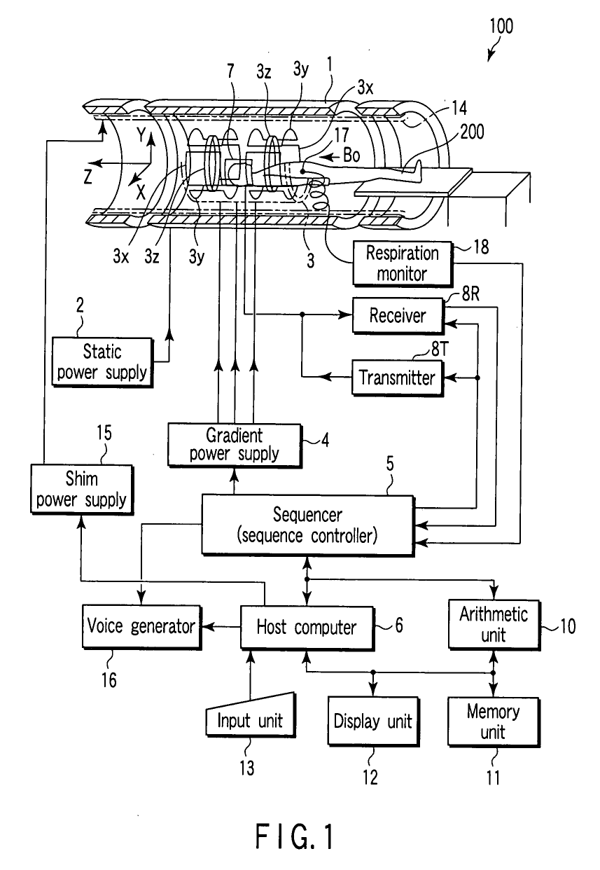

FIG. 1 is a view for showing a configuration of a magnetic resonance imaging apparatus (hereinafter, referred to as MRI apparatus) 100 related to the present embodiment.

The MRI apparatus 100 comprises a bed, a static magnetic field generating section, a gradient magnetic field generating section, a transmit and receive section, a control/computing section, a respiration measuring section, and an instructing section. The bed on which a subject 200 is positioned. The static magnetic field generating section generates static magnetic field. The gradient magnetic field generating section adds position information to the static magnetic field. The transmit and receive section transmits and receives radio frequency signals. The control/computing section controls the entire system and assumes image reconstruction. The respiration measuring section measures a respiratory signal synchronized with respiration of the subject 200. The instructing section gives various instructions to the subject 200. As components of each of these sections, the MRI apparatus further comprises a magnet 1, a static power supply 2, a gradient coil unit 3, a gradient power supply 4, sequencer (sequence controller) 5, a host computer 6, an RF coil unit 7, a transmitter 8T, a receiver 8R, a arithmetic unit 10, a memory unit 11, a display unit 12, an input unit 13, a shim coil 14, a shim power supply 15, a voice generator 16, a respiration sensor 17 and a respiration monitor 18.

The static magnetic field generating section includes the magnet 1 and the static power supply 2. For example, a superconducting electromagnet or a normal conducting electromagnet can be used as the magnet 1. The static power supply 2 supplies an electric current to the magnet 1. Hence, the static magnetic field generating section generates a static magnetic field B₀ in a cylindrical space (a space for diagnosis) into which the subject 200 is sent. The magnetic field direction of this static magnetic field B₀ is approximately consistent with the axial direction (Z axis direction) of the diagnostic space. The static magnetic field generating section is further provided with the shim coil 14. This shim coil 14 is supplied with electric current from the shim power supply 15 under the control of the host computer 6 to generate a correcting magnetic field to homogenize the static magnetic field.

The bed slides the top board onto which the subject 200 is placed into and out of the diagnostic space.

The gradient magnetic field generating section includes the gradient coil unit 3 and the gradient power supply 4. The gradient coil unit 3 is arranged on the inner side of the magnet 1. The gradient coil unit 3 comprises three pairs of coils 3x, 3y and 3z to respectively generate gradient magnetic fields in the directions of an x-axis, y-axis and z-axis, which are mutually orthogonal. The gradient power supply 4 supplies a pulse current to the coils 3x, 3y and 3z under the control of the sequencer 5 to generate the gradient magnetic field. By controlling the pulse current supplied from the gradient power supply 4 to the coils 3x, 3y and 3z in this manner, the gradient magnetic field generating section combines the gradient magnetic fields of physical axes in the three axial (X axis, Y axis and Z axis) directions, and arbitrary sets each gradient magnetic field for each logic axial direction comprised of a slice direction gradient magnetic field Gss, a phase encode direction gradient magnetic field Gpe, and a read out direction (frequency encode direction) gradient magnetic field Gre, which are mutually orthogonal. Each of the gradient magnetic fields Gss, Gpe and Gre in the slice direction, the phase encode direction and the read out direction are superposed on the static magnetic field B₀.

The transmit and receive section includes the RF coil unit 7, the transmitter 8T and the receiver 8R. The RF coil unit 7 is arranged near the subject 200 in the diagnostic space. The transmitter 8T and the receiver 8R are connected to the RF coil unit 7. The transmitter 8T and the receiver 8R are operated under the control of the sequencer 5. The transmitter 8T supplies an RF current pulse of Larmor frequency to the RF coil unit 7 to cause nuclear magnetic resonance (NMR). The receiver 8R captures an MR signal (radio frequency signal), such as an echo signal received by the RF coil unit 7, applies various signal processing, such as preamplifying, intermediate frequency conversion, phase detection, low frequency amplification or filtering, thereto, and generates echo data (original data) in a digital amount corresponding to the echo signal by A/D conversion. The RF coil unit 7 is a multi-coil embedded with a plurality of element coils. The receiver 8R is capable of processing the echo signal received by each of the plurality of element coils, in parallel.

The control/computing section includes the sequencer 5, the host computer 6, the arithmetic unit 10, the memory unit 11, the display unit 12 and the input unit 13.

The sequencer 5 comprises a CPU and a memory. The sequencer 5 stores pulse sequence information sent by the host computer 6 in the memory. The CPU of the sequencer 5 controls the operations of the gradient power supply 4, the transmitter 8T and the receiver 8R. The echo data output by the receiver 8R is once input to the sequencer 5, then transferred to the arithmetic unit 10. Here, the sequence information includes all information required to operate the gradient power supply 4, transmitter 8T and receiver 8R in accordance with the series of pulse sequence. Such information includes information related to, for example, strength of the pulse current to be applied to the coils 3x, 3y and 3z, application time and application timing.

The host computer 6 has various functions which can be realized by implementing predetermined software procedures. One of these functions is to give instructions of the pulse sequence information to the sequencer 5 and take control over the operation of the entire system.

Prior to imaging scan, the host computer carries out preparations, such as scanning for positioning. The imaging scan is a three dimensional (3D) scan or a two dimensional (2D) scan, which collects pairs of echo data required for image reconstruction. For the pulse sequence of the imaging scan, methods such as an spin echo (SE) method, fast spin echo (FSE) method, an fast asymmetric spin echo (FASE) method, which is the combination of a high speed method and a half Fourier method, an echo planar imaging (EPI) method and an fast field echo (FFE) method are used.

The echo data output by the receiver 8R is input to the arithmetic unit 10 via the sequencer 5. The arithmetic unit 10 allocates the input echo data to the k-space (also referred to as Fourier space or frequency space) set in the inner memory. The arithmetic unit 10 subjects the echo data allocated to the k-space to two dimensional or three dimensional Fourier conversion to reconstruct image data in real-space. Further, the arithmetic unit 10 is capable of performing, such as, synthesis processing or differential computation processing. Further, the arithmetic unit 10 comprises a function to perform reconstruction processing to realize parallel imaging based on methods such as sensitivity encoding (SENSE) and simultaneous acquisition of spatial harmonics (SMASH).

The synthesis processing includes a processing which adds image data of a plurality of two dimensional frames to each corresponding pixel, and a maximum projection (MIP) processing or a minimum projection processing, which selects a maximum value or a minimum value in the direction of the line of sight with respect to the three dimensional data. In addition, as another example of synthesis processing, the axis of a plurality of frames may be matched on the Fourier space and may be synthesized on an echo data of 1 frame in the form of an echo data. Further, the addition processing includes, for example, a simple addition, an addition averaging and a weighted addition.

The memory unit 11 stores the reconstructed image data as well as the image data having undergone the above synthesis processing and differential processing.

The display unit 12 displays various images to be presented to the user, under the control of the host computer 6. A display device, such as a liquid crystal display unit, can be used as the display unit 12.

The input unit 13 inputs a variety of information, which is related to, for example, imaging conditions, pulse sequence, image synthesis and differential computation required by the operator. The input unit 13 sends the input information to the host computer 6. The input unit 13 is provided arbitrary with a pointing device, such as a mouse or a track ball, a selective device, such as a mode selection switch, or an input device, such as a keyboard.

The instructing section comprises a voice generator 16. The voice generator 16 can set forth various messages by voice under the command of the host computer 6.

The respiration measuring section includes the respiration sensor 17 and the respiration monitor 18. The respiration sensor 17 is attached to the body surface of the subject 200, detects the abdominal movement of the subject 200, and generates a respiration signal showing the respiration condition of the subject 200. The respiration monitor 18 subjects the respiration signal output from the respiration sensor 17 to various processing including digitalization processing, and outputs such signal to the sequencer 5 and the host computer 6. The sequencer 5 uses the respiration signal when carrying out the imaging scan.

An operation of the MRI apparatus 100 configured in the above manner will be explained in detail as follows.

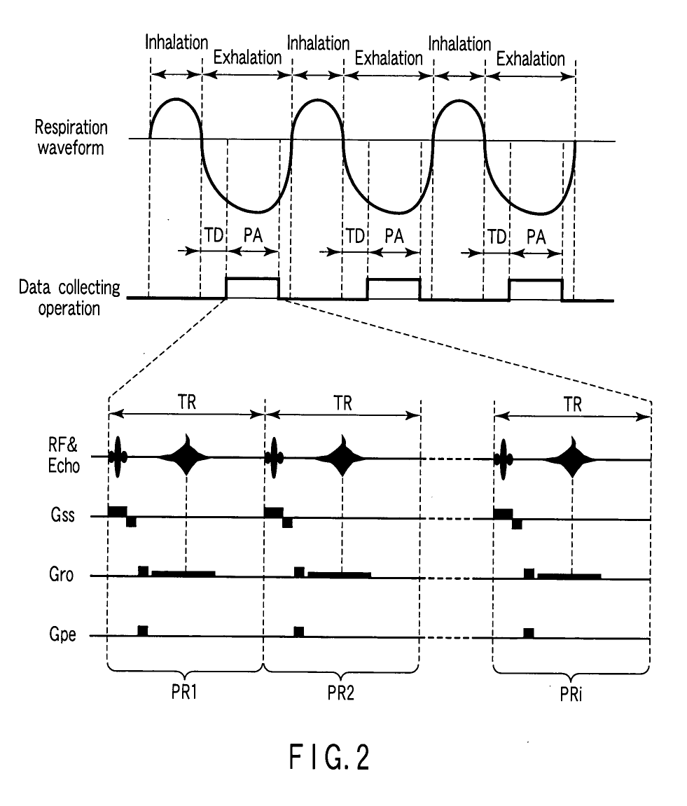

FIG. 2 is a view for showing timing to implement data collecting operation and a pulse sequence for collecting data.

A waveform shown in the uppermost part of FIG. 2 is the waveform of the respiration signal (referred to as respiration waveform hereinafter) output by the respiration sensor 17. As shown in FIG. 2, the sequencer 5 carries out data collecting operation for one slice during the collecting period PA within one exhalation period. Further, the sequencer 5 determines the start of the collecting period PA as the time point when a predetermined delay time TD has passed from the start of the exhalation period. The delay time TD may be a fixed value, or may be determined arbitrary based on the respiration waveform.

As shown in FIG. 2, the sequencer 5 controls, for example, the gradient power supply 4, the transmitter 8T and the receiver 8R, so that the data collecting operation is performed by a pulse sequence according to the FFE method. The waveforms of the pulse sequence shown at the bottom of FIG. 2 show, from top, a radio frequency pulse (RF) applied to the subject and echo (Echo) occurred in the subject, slice direction gradient magnetic field waveform (Gss), read out direction gradient magnetic field waveform (Gro), and phase encode direction gradient magnetic field waveform (Gpe).

In one collecting period PA, a similar sequence is repeated over i times in certain repeating times TR. The sequencer 5 controls the gradient power supply 4 so that a phase encode gradient is varied sequentially in each of the i pieces of periods PR1, PR2, . . . , PRi. However, the amount of change in the phase encode gradient is set so as to skip a part (for instance, ½) of the plurality of phase encode steps which is determined according to the desired field of view (FOV).

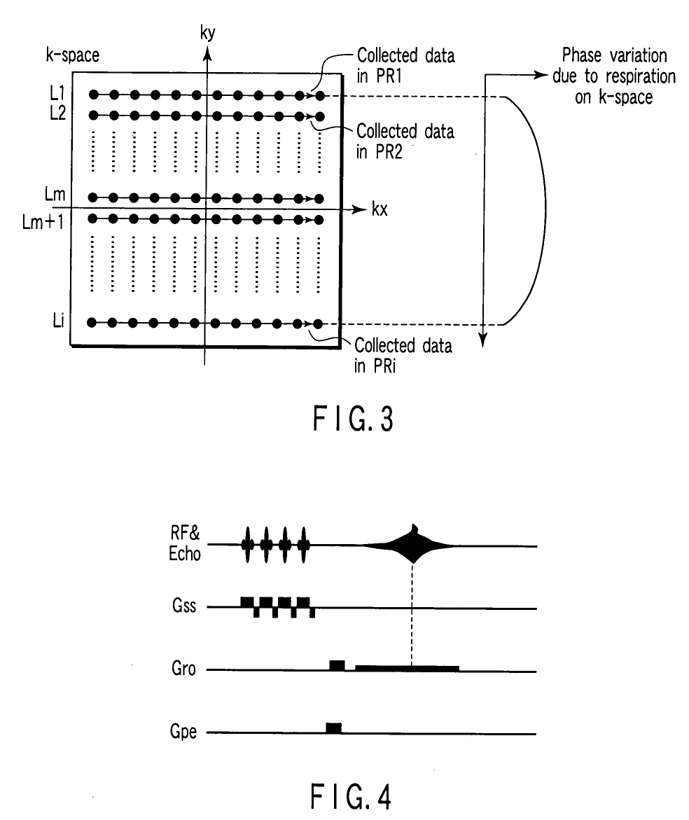

As shown in FIG. 3, the sequencer 5 allocates the data obtained in each of the periods PR1, PR2, . . . , PRi to the phase encode lines L1, L2, . . . , Li of the k-space, sequentially.

The collecting operation as mentioned above is carried out in parallel with respect to the echo signals received respectively by at least two element coils among the plurality of element coils embedded in the RF coil 7. The arithmetic unit 10 carries out image reconstruction using an algorithm for parallel imaging, such as the SENSE algorithm, based on the collected data.

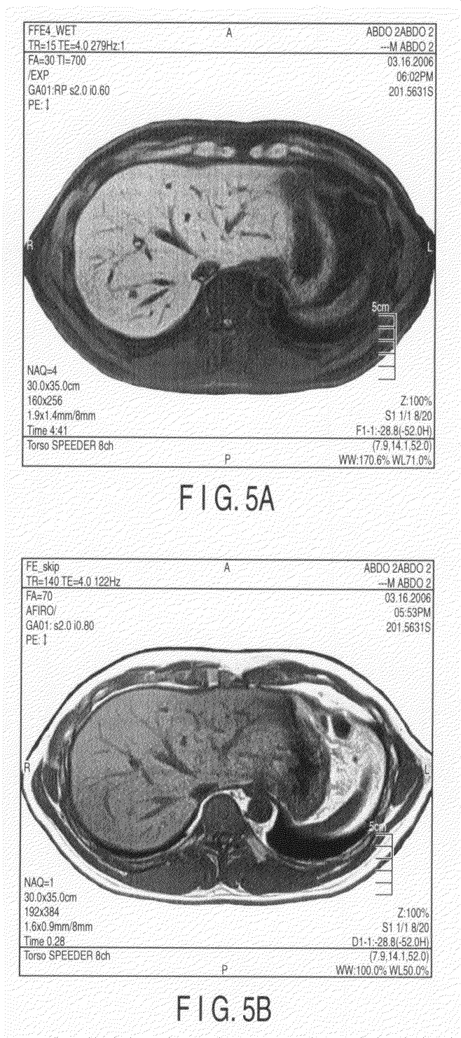

Meanwhile, the sequence in each of the periods PR1, PR2, . . . , PRi can be changed to, for example, a sequence according to the FFE method, which simultaneously uses a water excitation method, as shown in FIG. 4. FIG. 5A shows an example of an image obtained by employing the sequence shown in FIG. 4. FIG. 5B shows an example of an image obtained by a conventional method, i.e. breath-holding imaging, in a pulse sequence according to an field echo (FE) method. Further, the number of water excitation pulses can be varied arbitrarily.

The water excitation method is one of the methods which utilizes the difference of excitation frequency between water and fat and controls the magnetic resonance signal from the fat by mainly exciting the proton of water. In this water excitation method, a binomial RF pulse is used as a flip pulse.

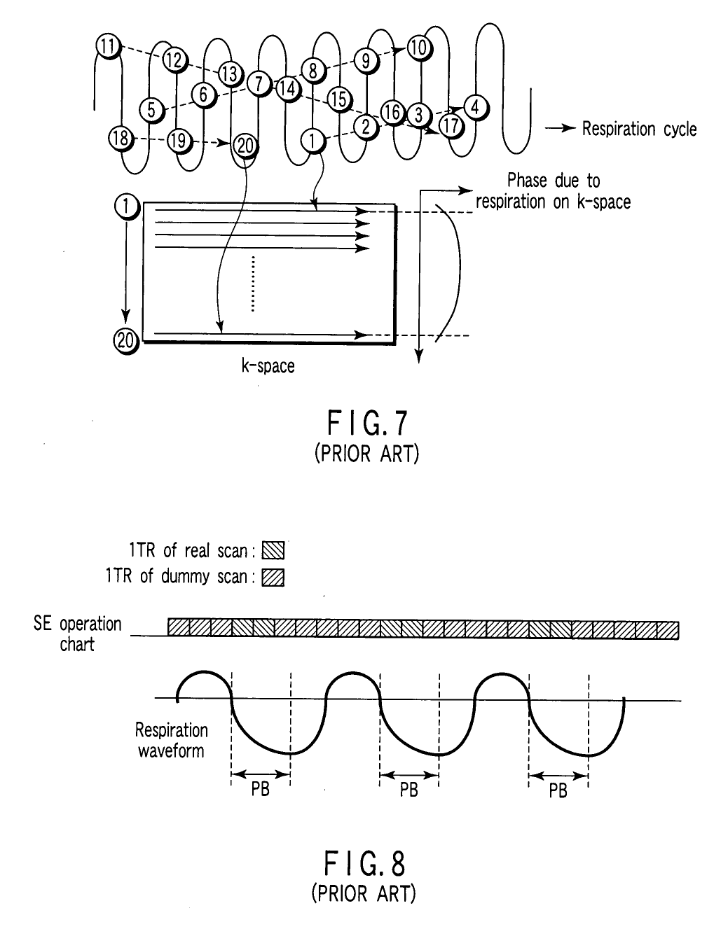

Thus, according to the present embodiment, a slice of data is collected within an exhalation period. Therefore, by allocating the data to be allocated to each phase encode line in the k-space sequentially in the sequence order of the phase encode line, it is possible to arrange the data in the k-space in the order of less phase change caused by respiration, without having to carry out any particular correction or rearrangement as shown in FIG. 7. By carrying out reconstruction based on the data allocated to the k-space in this manner, a fine image with reduced movement artifact can be obtained. Additionally, since it is also possible to improve S/N easily by increasing Nex (average), a clinically effective image can be obtained. Further, for instance, in the case of considering performing abdominal imaging in a strong static magnetic field of about 3 tesla (3T), it is assumed that the FE method or FFE method, which has low specific absorption rate (SAR), will most frequently be used. Therefore, by using the present approach, the SAR can be further reduced in comparison to using the SE method.

According to the present embodiment, a part of the plurality of phase encode steps is skipped by carrying out parallel imaging. Therefore, the collecting period PA can be shortened, and the collection of a slice of data can be completed unfailingly within an exhalation period.

According to the present embodiment, the collecting operation starts at time point when after the pass of the delay time TD from the timing of the start of exhalation period. Therefore, the collecting period can be implemented during a period having lesser movement in the exhalation period, which allows for further stabilized imaging. Meanwhile, it is possible to increase the number of collected PE data in a certain imaging time by increasing the number of channels of the coil and improving the increasing rate of imaging speed. As a result, it is possible to seek improvement in resolution.

According to the present embodiment, the simultaneous use of the water excitation method can bring about fat suppression effect without being influenced by a segment division. In the case of a CHESS (chemical shift selective) method, the length of fat control pulse is too long to apply to the present embodiment. Meanwhile, the water excitation method is suitable for the present embodiment since it is capable of keeping down the extension of the repeating time TR.

According to the present embodiment, since there is less influence from movement, a ghost is not emphasized by averaging. Therefore, it becomes easier to control the S/N.

This embodiment is capable of implementing various modified embodiments as follows.

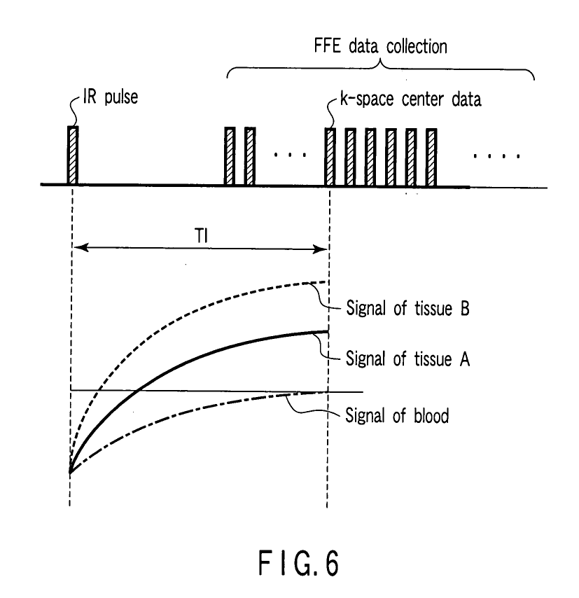

As shown in FIG. 6, an inversion recovery (IR) pulse may be applied prior to collecting data in the sequence shown in the above embodiment. A T1W image can be thus obtained while the movement artifact is suppressed. Further, it is possible to suppress the signal generated from blood while maintaining the contrast of T1W, by adjusting the IR pulse power (angle) and time TI from which the IP pulse is applied until the k-space center data is collected, so that the blood signal becomes null.

The present embodiment can also be adapted to three dimensional imaging carried out by the FFE method (FFE3D). Application of the present invention to the three dimensional imaging will improve slice resolution and expand clinical range of application dramatically. In the case of FFE3D, it is possible to seek reduction in the movement artifact by arranging a trigger for each slice encode and collecting the PE encode portion likewise the above method. Further, it is preferred that each of the plurality of slice encodes in the three dimensional imaging are scanned sequentially in each of the successive respiration cycle.

In the case where the respiration cycle is long, it is also possible to collect data for a plurality of slices within a collecting period PA.

It is possible to collect data for a plurality of slices within a collecting period PA by increasing the speed rate of parallel imaging.

With regard to accumulation method, it is possible to simultaneously use addition in a raw data condition.

With regard to addition method, it is also possible to use real data obtained after fast Fourier transformation (FFT) to average addition.

In abdominal imaging, it is possible to satisfy both an attempt to suppress the blood signal while acquiring a T1 contrast by adjusting the time TI and the flip angle (FA) of the TI pulse. In order to acquire an image as shown in FIG. 5A in a static magnetic field of 1.5 tesla, the combination of TI=680 to 700 msec and FA=140 to 170 degrees was most effective.

This is not limited to respiration synchronization, but can also be used with an electrocardiogram (ECG) to collect data for a slice in a time phase with less movement in tissues, such as heart.

Other than using IR, it is also possible to suppress the blood signal by simultaneously using an motion probing gradient (MPG) pulse.

Further, an realtime motion correction (RMC) technique and navigator echo can be used in combination to further suppress movement artifact. This prevents misalignment in the slice-section caused by movement of diaphragm and improves image viewability.

Data may also be collected within the inhalation period.

The increasing rate of parallel imaging speed designated by the user is input to the host computer 6 via the input unit 13. Based on this input increasing rate SR, the host computer 6 is capable of calculating the number of slices SN obtained within a collecting period PA using the following equation (1). Here, PE is the number of matrixes in the encode direction.

SN=PA/(TR×PE/SR) (1)

In the case of varying the collecting period in accordance with the respiration cycle of the subject 200, PA should also be varied in the above equation (1). Here, since the respiration cycle of the subject 200 varies frequently, the collecting period should also be varied in accordance with the respiration cycle. However, if the collecting period varies for each cycle, the collected signal may become unstable. Therefore, it is desirable that the collecting period be determined based on the average value, median value, maximal value or the minimal value of the respiration cycle in the plurality of cycles. The PA in the above equation (1) should also employ a value according to the collecting period determined as above. In the case where the repeating cycle or the number of matrixes in the encode direction is variable, a value which correspond accordingly is employed as TR or PE in the above equation (1).

The host computer 16 is capable of having the display unit 12 display the number of slices calculated by the above equation (1) to notify the user. By doing so, the user is notified of the number of slices which can be collected in a collecting period. In the case where the number of slices to collect in a collecting period is set in accordance with the user's instructions, this enables the user to designate an appropriate number of slices.

Further, the host computer 16 is capable of automatically setting the number of slices to be collected in a collecting period based on the number of slices calculated by the above equation (1).

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A magnetic resonance imaging apparatus comprising:
a collecting unit which collects magnetic resonance data regarding a subject by a predetermined pulse sequence; and
a control unit which controls the collecting unit so that it collects the magnetic resonance data for at least a slice within an inhaling or an exhaling period of the subject.

2. The magnetic resonance imaging apparatus according to claim 1, wherein the collecting unit collects the magnetic resonance data using a plurality of coils simultaneously while skipping a part of a plurality of phase encode steps, which is determined in accordance with a desired field of view.

3. The magnetic resonance imaging apparatus according to claim 2, further comprising a unit which reconstructs an image within the field of view with respect to the subject by parallel imaging, based on the magnetic resonance data collected by the collecting unit.

4. The magnetic resonance imaging apparatus according to claim 1, wherein the collecting unit employs an fast filed echo (FFE) sequence as the pulse sequence.

5. The magnetic resonance imaging apparatus according to claim 1, wherein the control unit controls the collecting unit so that it starts collecting the magnetic resonance data at a time point when a predetermined delay time passed from the start of an inhaling or an exhaling period.

6. The magnetic resonance imaging apparatus according to claim 1, wherein the collecting unit employs a sequence including a water excitation method which mainly excites water proton by using a binomial RF pulse for a flip pulse as the pulse sequence.

7. The magnetic resonance imaging apparatus according to claim 1, wherein the collecting unit transmits an inversion recovery (IR) pulse prior to collecting the magnetic resonance data by the pulse sequence.

8. The magnetic resonance imaging apparatus according to claim 7, wherein the collecting unit sets a flip angle of the IR pulse and period of time between which the IR pulse is transmitted and the magnetic resonance data to be allocated to center of k-space is collected, so as to suppress signals from bloodstream.

9. The magnetic resonance imaging apparatus according to claim 1, wherein the control unit controls the collecting unit so that it collects the magnetic resonance data for a plurality of slices within the inhalation or the exhalation period.

10. The magnetic resonance imaging apparatus according to claim 3 further comprising:
a setting unit to set increasing rate of imaging speed by the parallel imaging according to a user's assignment; and
a computing unit to compute the number of slices obtained within the inhalation or exhalation period, based on the increasing rate of imaging speed set by the setting means.

11. The magnetic resonance imaging apparatus according to claim 10, wherein the computing unit computes the number of slices obtained within the inhalation or the exhalation period in consideration with the user's respiration cycle, in addition to computing the increasing rate of imaging speed set by the setting means.

12. The magnetic resonance imaging apparatus according to claim 10, further comprising a display unit which displays the number of slices computed by the computing unit.

13. The magnetic resonance imaging apparatus according to claim 10, wherein the control unit controls the collecting unit so that it collects the magnetic resonance data for the number of slices computed by the computing unit within the inhalation or the exhalation period.

14. A magnetic resonance imaging apparatus comprising:
a collecting unit which collects magnetic resonance data regarding a subject by a predetermined pulse sequence; and
a control unit which controls the collecting unit so that it collects the magnetic resonance data for at least a slice within a period in which a heart of the subject has less variation in movement.

15. A magnetic resonance imaging apparatus comprising:
an obtaining unit which obtains a respiration signal which indicates respiration of a subject;
a scan unit which carries out a three dimensional scan of the subject in synchronization with the obtained respiration signal; and
a control unit which controls the scan unit so that it collects the magnetic resonance data for a slice encode of the three dimensional scan within an inhalation or an exhalation period based on the respiration signal.

16. The magnetic resonance imaging apparatus according to claim 15, wherein the control unit controls the scan unit so that it starts collecting the magnetic resonance data for each of the plurality of slice encodes of the three dimensional scan at a time point when a predetermined delay time passed from a reference time point of each cycle in each different cycle of the exhalation signal.
